(19) United States
(12) Patent Application Publication
(10) Pub. No.: US 2011/0230690 A1
(43) Pub. Date: Sep. 22, 2011

(54) PROCESS FOR OLIGOMERIZING OLEFINS

(76) Inventors: Marja Tiita, Porvoo (FI); Helka Turunen, Porvoo (FI); Kaija Isokoski, Porvoo (FI); Antti Pyhalahti, Helsinki (FI)

(21) Appl. No.: 12/672,064

(22) PCT Filed: Aug. 25, 2008
(86) PCT No.: PCT/FI08/50473

§ 371 Date: Feb. 3, 2010

(30) Foreign Application Priority Data
Aug. 24, 2007 (FI) 20075589

(51) Int. Cl. C07C 2/10; C07C 2/08

(52) U.S. Cl. 585/255; 585/510

(57) ABSTRACT

A process for oligomerizing olefinic, lower hydrocarbons. The process comprises the steps of feeding a fresh olefinic hydrocarbon feedstock to a reaction zone; contacting the olefinic hydrocarbons of the feedstock with an acidic catalyst in the reaction zone in order to dimerize at least a part of the olefinic hydrocarbons, withdrawing an effluent containing oligomerized olefins from the reaction zone; and conducting the effluent to a separation zone, wherein the oligomerization reaction product is separated from said effluent. According to the invention, the reaction is carried out in homogeneous phase comprising a solvent for olefinic hydrocarbons, maintained at supercritical conditions. By using supercritical carbon dioxide as a solvent deactivation rate of catalyst can be diminished. Carbon dioxide is easy to remove from the product mixture and spent reaction medium can be used for regeneration of the catalyst.

### BACKGROUND OF THE INVENTION

1. Field of the Invention

The present invention relates to the production of hydrocarbon oligomers, in particular dimers, useful as fuel components of combustion engines and as raw-materials of other hydrocarbon products. In particular, the present invention concerns a process for dimerizing and potentially oligomerizing lower, olefinic hydrocarbons in the presence of an acidic aluminosilicate catalyst.

2. Description of Related Art

Light olefin dimers are useful intermediates in the manufacture of different products, like alcohols, ketones and carboxylic acids. Highly branched trimethylolefins and trimethylparaffins and trimers of butenes are useful as gasoline octane number enhancers.

Many processes for utilization of light olefins for the production of high quality transportation fuels are known. The Mobil Olefin to Gasoline and Distillate (MOGD) process converts propylene and butylene to olefinic distillate in high yields. The MOG or Mobil Olefins to Gasoline process is an extension of the MOGD. In MOG, the reaction conditions allow aromatics formation.

Oligomerization of isobutene from C₄ olefins over zeolite catalysts has been disclosed in several U.S. patents. There are several processes based on ion exchange resins available, as described e.g. in U.S. Pat. Nos. 4,375,576 and 4,100,220. They have many good properties but they all have the drawback of being totally dependant on oxygenate moderator, which improves the selectivity. This moderator has to be recycled and there is usually the same amount of oxygen containing side products present in the dimerized product. When the reason to use dimerization is elimination of oxygenates from the components, these are highly undesired. Moreover, oxygenates make hydrogenation of the dimerized product more difficult.

The use of an acidic aluminosilicate catalyst will remove the need for oxygenates in dimerization/-oligomerization, and a high selectivity can be reached. One particular feature relating to the use of an acidic aluminosilicate catalyst is, however, the need for regeneration of the catalyst which leads to the use of several parallel reactors, at least one of which is constantly being placed in regeneration mode.

The limited operational times of the catalyst are caused by formation of coke in the pores of a catalyst during hydrocarbon conversion. If coke deposition rate exceeds the rate at which coke is removed from the catalyst surface, coke will eventually clog the pores. When oligomerization is operated in gas phase, coke removal is volatility driven. As a result, coke will not desorb from the surface of the catalyst because the coke is composed mainly of heavy hydrocarbon which have low volatility. Even in liquid phase oligomerization, where coke removal is solubility-driven, there is only limited diffusion of coke from the pores to the solvent.

The deactivated acidic aluminosilicate catalysts are usually regenerated at high temperature of 500-800° C. with air. The purpose of catalyst regeneration is to burn off coke formed on the catalyst surface because the coke decreases the activity of the catalyst. The objective is typically to burn all or most of the coke in the catalyst. Oxidizing agents, such as ozone and nitrous oxide, have also been used for the elimination of coke from zeolites. Oxidative treatments often has detrimental effects on the active sites of the catalyst and the choice of operation conditions is important in limiting the undesired effects which particularly water has on the active sites of the catalysts at high temperature.

### SUMMARY OF THE INVENTION

It is an object of the present invention to eliminate at least a part of the problems of the prior art and provide a novel process for dimerizing olefinic feedstocks.

In particular, it is an object of the present invention to provide a new acidic aluminosilica-catalysed oligomerization process which can be operated at technically feasible times while avoiding excessive regeneration of the catalyst.

The invention is based on the idea of oligomerizing C₃ to C₅ olefins in homogeneous phase, which may be formed by a liquid or supercritical fluid, in a reaction sequence comprising at least one reaction zone and at least one separation zone. The reaction is carried out at conditions in which at least a part of the olefins oligomerize. The separation zone is arranged after the reaction zone, and a circulation flow is circulated from the separation zone back to dimerization. The process is carried out essentially in the absence of polar compounds.

According to the invention, an olefinic feedstock comprising C₃ to C₅ isoolefins is contacted with an acidic aluminosilica catalyst in homogeneous phase in order to dimerize/oligomerize the isoolefins into C₆ to C₁₀ dimers and corresponding trimers. Surprisingly, it has been found that the use of a solvent comprising or even consisting of pressurized gas like supercritical carbon dioxide during oligomerization delays deactivation and extends run periods in oligomerization of lower isoolefins, such as isobutene.

A further advantage comes from the finding is that supercritical carbon dioxide is also a suitable regeneration medium for deactivated catalyst at low temperatures (at temperatures below about 500° C.).

The catalyst can be any acidic aluminosilica catalyst that is active in dimerization reactions. Such acidic aluminosilica catalyst are exemplified by natural and synthetic zeolites, for example medium pore size zeolites, such as ZSM-5, ferrierite, ZSM-22 and ZSM-23, large pore size zeolites, such as beta or Y-zeolite, amorphous silica alumina catalysts such as MCM-41 or clays.

The invention also provides the use of a synthetic or natural zeolite and amorphous mesoporous aluminosilicates as an acid catalyst for dimerization of an olefinic feed containing unsaturated hydrocarbons, selected from the group consisting of isobutene, 1-butene, 2-butene, linear C₅ olefins and branched C₅ olefins in liquid phase employing a supercritical solvent.

More specifically, the process according to the present invention is mainly characterized by what is stated in the characterizing part of claim 1.

The use according to the invention is characterized by what is stated in the characterizing part of claim 35.

Considerable advantages are achieved by means of the present invention. The use of a pressurized gas as solvent, in particular carbon dioxide at supercritical state or use of expanded solvents, is very beneficial since carbon dioxide is easy to remove from the product mixture and the use of carbon dioxide as a reaction medium is very fire safe and otherwise safe, too. The carbon dioxide utilization also gives an opportunity for decreasing emissions of carbon dioxide into the atmosphere,

Further, high selectivity of dimers can be achieved thus making the production more efficient compared with previously used processes.

With conventional techniques, regeneration of the catalyst often poses a threat to continuous operation of the process. In the present invention, the catalyst can be regenerated continuously, during process operation. The easy regeneration gives a possibility to handle feeds containing nitrogen and sulphur impurities, which is a considerable advantage compared to previous processes.

In prior art, it is common to operate dimerization processes in the presence of oxygenates or other polar compounds. Oxygenates and other polar compounds typically include water, ether or alcohol. Alcohols commonly used in dimerization processes include C₁ to C₅ alcohols, e.g. methanol, ethanol, isopropanol or t-butanol. The alcohol may be primary, secondary or tertiary alcohol. Further examples include tert-amyl methyl ether, 2-butanol and 2-pentanol. According to the present invention, no oxygenates or polar compounds are required. This is a considerable advantage, because the separation of oxygenates from the dimerized product often poses problems in process design and operation. The design of process equipment can be simplified with no need for separation of oxygenates from the product flow. This means a simplified and more easily controlled process operation and savings in process equipment investments.

Additionally, as indicated above, the pressurized gas, like supercritical carbon dioxide, can also act as a regeneration medium for the catalyst. When the regeneration is performed with the same solvent to the reaction solvent, the hydrocarbons (feed, product and coke) in catalyst can be easily utilized, and no feed or product molecules are lost by burning.

At the conditions of the process, separation of the solvent is facile: it can be carried out by lowering the pressure to vaporize the gas forming the supercritical solvent. Also the separation of unreacted components from liquid phase is relatively easy.

### BRIEF DESCRIPTION OF THE DRAWINGS

FIG. 1 depicts in a schematic fashion the process configuration of the basic technical solution of the invention;

FIG. 2 depicts an embodiment in which the reaction zone comprises two reactors in parallel;

FIG. 3 depicts an embodiment in which the reaction zone comprises a fluidized bed reactor with continuous catalyst regeneration; and

FIG. 4 depicts an embodiment in which the reaction zone comprises 2 reaction zones and 2 separation zones.

### DETAILED DESCRIPTION OF PREFERRED EMBODIMENTS

### Definitions

In the present invention, the term “acidic aluminosilicate” covers various synthetic and natural zeolites and mesoporous silica aluminas capable of acting as acid catalysts in the present dimerization/oligomerization reaction of lower isoolefins, such as isobutene.

A “reaction zone” comprises at least one, typically two or three, reactor(s). The reactor can be any continuous type reactor, in which a solid catalyst can be placed and that is capable of handling liquid reagents. Advantageously, the reactor is a simple tubular reactor, a packed bed reactor or a fluidized bed reactor. The reactor can be a tubular reactor with multiple pipes, wherein the pipes are filled with catalyst. Other possibilities include a reactive distillation unit with side reactors. The operating pressure of the reactors depends on the type of the reactor and on the composition of the feed, typically it is desired to keep the reaction mixture in liquid phase. In order to be able to regenerate the catalyst during reactor operation, it is often advantageous to use at least two reactors that can be regenerated in turn. Another advantageous mode of operation is to use a reactor, in which the catalyst can be regenerated continuously.

For the purposes of the present invention, “separation zone” designates a separation system capable of separating the products from unreacted reactants and solvent. The separation system can comprise one or several separation units. In a simple embodiment, the separation system may comprise a vessel for reducing the pressure which allows for phase separation into the liquid and gas phase. According to an embodiment the separation system may comprise a distillation system with one or more distillation columns. The feed plate can be selected for each column to be most advantageous in view of the overall process. The distillation column can be any column suitable for distillation, such as a packed column, or one provided with valve, sieve or bubble-cap trays. The separation zone may comprise also absorption, adsorption, membrane separation or extraction stages.

“Isooctene” and “di-isobutene” are both products of isobutene dimerization. Thus they can be used interchangeably to designate 2,4,4-trimethyl-1-pentene and 2,4,4-trimethyl-2-pentene or a mixture thereof.

“Isooctane” and “di-isobutane” comprise the corresponding hydrogenated paraffinic compounds.

“Effluent” contains the desired product of the dimerization reaction in the reaction zone. When only C₃ olefins, only C₄ olefins or only C₅ olefins are fed to the process, it is clear that the resulting product of the mutual reactions of the olefins yield dimers. However, when C₃, C₄ and C₅ olefins are present in the feed, in addition to dimerization, also reactions between the different olefins may occur. The word “dimer” is also used for the reaction products in the specification for reasons of simplicity, but it is to be understood that when different lower olefins are present in the feed, the reaction mixture typically contains also some amount of the C₉ olefins.

As well-known in the art, materials can be present in solid, liquid or gas phase or in “supercritical state”. In the triple point of material the state of material changes between solid, gas and liquid phase. In the critical point of a material the state of material changes between liquid, gas and supercritical phase. The supercritical fluid exhibits weak surface tensions which are utilized in the present invention. These weak surface tensions promote the diffusion of fluid to the pores of the catalyst. The supercritical fluid used in the invention also has a dissolving power. As a result, deactivation of the catalyst can be restricted when the rate of coke formation is smaller than the rate of coke removed by the fluid from the pores.

### The Overall Process

According to the present invention, a hydrocarbon feed containing isobutene or linear butenes or a mixture thereof, is contacted with an acidic catalyst together in a essentially oxygenate-free reaction system comprising at least one reaction zone and at least one separation zone. The conditions in said reaction zone are essentially oxygenate-free, which means that the amount of polar compounds is less than 0.5 mole-% of the olefinic hydrocarbons fed into the reaction zone. A solvent in supercritical condition is present. The solvent is preferably different from the olefin or the starting material. In particular, a solvent comprising supercritical carbon dioxide is fed into the reaction zone.

The conditions in the reaction zone are such that at least a part of the isobutene is dimerized to isooctene. The flow from said reaction zone is introduced into a separation zone, where the main part of the dimerized reaction product is separated from the unreacted product. Apart from dimerization, usually some oligomerization takes place.

Advantageously, at least a part from the unreacted product along with the solvent is circulated from the separation zone back to oligomerization.

Thus, according to a preferred embodiment, a process according to the invention for oligomerizing olefinic, lower hydrocarbons, comprises the steps of
feeding a fresh olefinic hydrocarbon feedstock to a reaction zone;
contacting the olefinic hydrocarbons of the feedstock in homogeneous phase with an acidic catalyst in the reaction zone in order to oligomerize at least a part of the olefinic hydrocarbons, wherein the acidic catalyst is selected from the group of natural and synthetic zeolites and mesoporous aluminosilicates;
withdrawing an effluent containing oligomerized olefins from the reaction zone; and
conducting the effluent to a separation zone, wherein the oligomerization reaction product is separated from said effluent.

The feed of the process according to the present invention is a hydrocarbon mixture containing olefins. The feed comprises olefins to be dimerized at least 10 wt-%, preferably at least approximately 20 wt-%. As already described, the olefins are selected from the group of propene, linear 1- or 2-butene, isobutene and linear or branched C₅ olefins. Alternatively, the feed can comprise a mixture of any or every of the olefins listed above. Typically, the feed comprises dimerizable components; either C₄ olefins, preferably isobutene, whereby isooctene is produced, or C₅ olefins, whereby substituted C₁₀ olefins are produced. It is clear that both C₄ and C₅ olefins can be present in the feed, whereby a great variety of products is produced. The composition of the product flow is discussed later.

According to the first preferred embodiment, in which C₄ hydrocarbons are dimerized, the hydrocarbon mixture in the feed comprises at least 10 wt-%, preferably at least approximately 15 wt-% isobutene. The feed can consist of pure isobutene, but in practice, the feedstock readily available comprises C₄ based hydrocarbon fractions from oil refining. Preferably, the feed comprises a fraction obtained from isobutane dehydrogenation, when the feed comprises mainly isobutene and isobutane and possibly small amounts of C₃ and C₅ hydrocarbons. Typically the feed then comprises 40 to 60 wt-% of isobutene and 60 to 40 wt-% isobutane, usually there is 5 to 20% less isobutene present than isobutane. Thus, the ratio of isobutene to isobutane is approximately 4:6 . . . 5:5.5. As an example of an isobutane dehydrogenation fraction, the following can be presented: 45 wt-% isobutene, 50 wt-% isobutane and other inert C₄ hydrocarbons and approximately 5 wt-% of C₃, C₅ and heavier hydrocarbons altogether.

Due to the high isobutene content in the flow from the isobutane dehydrogenation the amounts of inert hydrocarbons in the recycling flows remain relatively small. The dehydrogenation fraction is very suitable for producing a product with a very high content of the dimerized isobutene.

The feed for producing isooctene is also possible to select from the group containing C₄ fractions of FCC, TCC, DCC and RCC or from the C₄ fraction after the removal of butadiene, also called Raffinate 1 of an ethylene unit. Of these FCC, RCC, TCC and Raffinate 1 are preferred, since the hydrocarbon fractions can be used as such, possibly after removing the heavier (C₈₊) fractions. Raffinate 1 is typically composed of approximately 50 wt-% isobutene, approximately 25 wt-% linear butenes and approximately 25 wt-% paraffins. The product from the FCC is typically composed of 10 to 50, in particular 10 to 30 wt-% isobutene, 20 to 70 wt-% 1- and 2-butene and approximately 5 to 40 wt-% butane. As an example of a typical FCC-mixture, the following can be presented: approximately 17 wt-% isobutene, approximately 17 wt-% 1-butene, approximately 33 wt-% 2-butene and approximately 33 wt-% butane, and others.

Also isobutene prepared from chemicals can be used as feed.

According to another preferred embodiment of the invention, the olefins present in the olefinic feedstock are selected from the group of linear and branched C₅ olefins, such as linear pentene, 2-methyl-1-butene, 2-methyl-2-butene, 3-methyl-1-butene, and mixtures thereof.

According to an embodiment of the invention, the feedstock comprises aromatic hydrocarbons, paraffins and mixtures of these.

If the present invention is used for converting linear butenes, the linear butenes are preferably selectively isomerized to 2-butene as completely as possible. In this case, it is preferable to add a separate side reactor circulation to the process configuration. The temperature in this reactor is preferably higher than in the prereactor or circulation reactor in order to increase the conversion of dimerization.

FCC and corresponding hydrocarbon flows are suitable to use, e.g., in cases where the conventional MTBE unit is used to produce a product mixture comprising isooctene and MTBE.

According to the second preferred embodiment of the invention, in which C₅ olefins are dimerized, the feed comprises olefins selected from the group of linear and branched C₅ olefins, or a mixture thereof. Thus, the olefins typically present in the feed comprise linear pentene, 2-methyl-1-butene, 2-methyl-2-butene, 3-methyl-1-butene. Also some amounts of C₆ olefins, typically at least 5 wt-% can be present in the feed.

According to a second preferred embodiment, the feed comprises FCC gasoline, light FCC gasoline, pyrolysis-Cₛ gasoline, TCC gasoline, RCC gasoline and Coker gasoline, typically the C₅ fraction of FCC gasoline, and can thus comprise also some C₆ olefins. Advantageously, the FCC fraction is fractionated to obtain as pure C₅ olefin fraction as possible where other C₅ hydrocarbons are present in less than 15 wt-%, preferably less than 5 wt-%. It is possible to use a fraction comprising also C₆ olefins. Typically, the feed then comprises 20 to 60 wt-%, in particular 30 to 50 wt-% C₅ olefins, 10 to 30 wt-%, in particular 15 to 25 wt-% C₆ olefins and 15 wt-% or less paraffinic hydrocarbons pentanes.

According to the third preferred embodiment, the feed comprises both C₄ and C₅ olefins. In this case, the feed is typically selected from the group comprising FCC, TCC, DCC and RCC or from the C₄ fraction after the removal of butadiene, also called Raffinate 1 of an ethylene unit, FCC gasoline, light FCC gasoline, pyrolysis-C.sub.5-gasoline, TCC gasoline, RCC gasoline and Coker gasoline. A fraction readily available comprises C₄ and C₅ fractions from FCC. Advantageously, a fraction comprising at least 10 wt-%, preferably at least 15 wt-% C₄ olefins and at least 10 wt-%, preferably at least 15 wt-% C₅ olefins is used. Typically the amounts of C₄ olefins and C₅ olefins are approximately equal, although a slight dominance of C₄ olefins in the fraction is also usual.

According to the invention, the hydrocarbon feed containing olefins is contacted with an acidic catalyst selected from the group of natural and synthetic zeolites and mesoporous aluminosilicates together in a reaction zone at conditions in which at least a part of the olefins is dimerized. In case where the olefin feed comprises C₃ to C₅ olefins, also reactions between different olefins occur, thus forming higher (meaning up to C₁₀) olefins. Furthermore, some trimers are usually formed. The effluent from the reaction zone is introduced into a separation zone, where the main part of the dimerized/oligomerized reaction product is separated.

The dimerization/oligomerization reaction is carried out in one phase. In particular the reactants and the solvent are kept at supercritical conditions or near critical conditions. It has been surprisingly found that with acidic catalysts of the above kind, the use of a solvent maintained in supercritical phase is advantageous, because it has the capacity of a liquid solvent to dissolve and leach coke from the surface and that of a gaseous substance to extract coke out from the pores of the catalyst. The run time of the catalyst can be prolonged significantly, from at least two times the normal time between subsequent regenerations up to 10 to 20 times longer.

Generally, the solvent can be selected from the group consisting of nitrogen, methane, trifluoromethane, carbon dioxide, ethane, nitrooxidule, sulphur hexafluoride, difluoromethane, ammonia, propane, isobutene and water and mixtures thereof. A particularly preferred solvent comprises carbon dioxide having a purity of about 50 to 100% by weight. The solvents could also be expanded solvents.

The critical point of carbon dioxide is 31° C. (i.e. about 304 K) and 73 bar. Such conditions are feasible in the industrial operations. Carbon dioxide is also non-toxic, non-flammable and do not easily react with hydrocarbons. Besides exhibiting dissolving power and weak surface tensions, these properties are good for the use of a substance as a solvent.

The use of supercritical carbon dioxide as a solvent in reactions is known for several reactions. Supercritical carbon dioxide has been studied as a solvent for alkylation reactions of isobutane with 1-butene, of toluene with propylene, and of benzene with ethene with different zeolite catalysts. Thus, Published PCT Patent Application WO 2006/128649 discloses a process carried out at superatmospheric pressure with nitrogen for rejuvenating catalysts which have been deactivated by use in liquid-phase or supercritical or dense-phase olefin oligomerization, or by use in aromatic alkylation. It has also been utilized in the preparation of dimethyl carbonate and hydrogenation.

Now, we have found that the supercritical carbon dioxide is an excellent solvent for the dimerization/oligomerization reaction of C₃-C₅ olefins because it limits the deactivation rate of acidic aluminosilica catalysts. Additionally, we have also found that supercritical carbon dioxide as a regeneration medium is capable of activating the deactivated catalyst at a low temperature (below 300° C./about 570 K).

During the dimerization reaction, the olefinic hydrocarbons and the solvent are maintained at a pressure (in particular absolute pressure) of 15 to 200 bar (1.5 MPa to 20 MPa) and the temperature of the homogeneous phase is 300-420 K.

The concentration of the solvent with respect to the hydrocarbon feed can vary freely. Typically, solvent is used in a molar ratio of 100:1 to 1:100 with respect to the feed, particularly preferred molar ratios are 10:1 to 1:10, in particular about 8:1 to 1:1.

The hydrocarbon feed is mixed with the solvent either in the reactor by utilizing the good solubility of the feed or by using a mixer or agitator. It is also possible to contact the feed with the solvent by combining the feed streams of the hydrocarbons and of fresh solvent feed/recirculation solvent feed in an inlet to the reactor. Optionally, mixing can be improved with a static mixer.

A part of the first reaction product is circulated from the separation zone back to the reaction zone. It is to be understood that although the following description refers to a sideflow in the singular tense, which is the typical configuration, it is also possible to withdraw two or more side flows and circulate all those flows back to dimerization.

According to an advantageous embodiment, the reaction zone comprises two reactors in parallel. The feed comprising fresh olefinic feed and recycled first product may be fed to one of the reactors, and the second reactor can be subjected to catalyst-regeneration simultaneously. The effluent from the reaction zone is introduced into a separation zone, where the main part of the dimerized reaction product is separated to form a first product containing unreacted hydrocarbons and a second product containing the dimerized olefins.

The flow of the recycled stream from the separation zone, in particular from a first unit of a multistage separation system, is 20 to 150 wt-%, preferably 30 to 130 wt-%, in particular 40 to 120 wt-% of the flow of the fresh feed.

The selectivity of the dimerization reaction in a process according to present invention is high. According to an embodiment, the selectivity of dimerized olefins, expressed as the ratio of the molar amount of dimeric compounds to the total molar amount of converted olefins, is in excess of 0.8, in particular in excess of 0.9.

According to an advantageous embodiment, the reactor is a reactive distillation unit with side reactors.

The catalyst of our invention can be regenerated. This makes it possible to work in a continuous process with two reactors in that the one is in a reaction stage and the other is in the regeneration stage. This gives the opportunity to handle a feed with a high level of nitrogen and sulphur impurities that deactivates the catalysts.

In practice, regeneration is carried out by heating the spent catalyst to an elevated temperature in the presence of oxygen to burn off coke and other impurities which have gathered on the surface of the catalyst or inside the pores thereof. Typically, the temperature is higher than 500° C.

As already indicated above, for the regeneration, the used, i.e. spent reaction solvent, such as carbon dioxide at supercritical condition in reactor inlet, can also be used as a regeneration medium. It has been found that when the regeneration is performed with the same solvent used as reaction solvent, the hydrocarbons (feed, product and coke) in catalyst can be easily utilized, and no feed or product molecules are lost by burning. Thus, according to a preferred embodiment, the catalyst is regenerated by heating it in a regeneration medium comprising 50 to 100% supercritical carbon dioxide, said percentage being calculated from the weight of the medium.

According to this embodiment, the regeneration can be carried out at temperature below 500° C., in practice regeneration can be operated at about 100 to 400° C., preferably between 200 to 300° C.

Supercritical carbon dioxide used as reaction or regeneration medium can be separated with a flash-type separation system.

The effluent from the reaction zone is conducted to a separation zone, where components are separated from one another. The composition of the product flow depends on the process parameters and on the composition of the feed. As already discussed, the process of the present invention can be used for producing dimerized product from olefinic feedstock. The olefins present in the feed can be either C₃ olefins, C₄ olefins, C₅ olefins or a mixture of these. Thus it is clear that the composition of the product flow depends essentially on the fraction used as the feedstock.

According to the invention, an acidic catalyst is used for dimerization/oligomerization. In these conditions, natural and synthetic zeolites or mesoporous aluminosilicates are active and selective for trimethylolefins. The catalyst can be 10-member ring zeolites such as ZSM-5, ZSM-22 or ZSM-23, or 12-member ring zeolites such as beta or Y-zeolite. The catalyst can also be mesoporous aluminosilicate having a regular pore system such as MCM-41 or amorphous mesoporous aluminosilicate having an irregular pore system.

According to one preferred embodiment, the zeolite is selected from the group consisting of synthetic and natural zeolites containing about 0.1 to 5 wt-%, preferably about 0.3 to 3 wt-%, in particular about 0.5 to 2 wt-%, aluminium. The zeolite is selected from the group consisting of ZSM-5, ZSM-22, ZSM-23, ferrierite and ion-exchanged zeolites prepared therefrom, or from the group consisting of beta or Y-zeolite and ion-exchanged zeolites prepared therefrom. Such ion-exchanged zeolites may contain counter-ions selected from the group of alkali metal and alkaline earth metal ions, such as sodium, potassium, calcium and magnesium.

Zeolite catalysts used according to the invention can be prepared by any suitable method known in the art. A common method to prepare zeolites is preparation by hydrothermal synthesis. In hydrothermal synthesis, a reaction mixture containing a source of silicon oxide, a source of aluminium oxide and if necessary an organic template together with an alkali metal source are stirred together at appropriate temperature. The formed crystals are separated from the mixture, and calcinated in air at such temperatures and such a time that the organic template is removed. The ions of the calcinated material are exchanged to ammonium ions. The material is subjected to suitable conditions to decompose ammonium ions in order to form ammonia and protons.

According to another preferred embodiment, the catalyst is an amorphous mesoporous aluminosilicate which is selected from the group consisting of synthetic and natural aluminosilicates having a regular or irregular pore system. They contain aluminium about 0.1 to 50 wt-%, preferably about 2 to 10 wt-%. The BET surface areas of materials are between 200 and 1000 m²/g, preferably between 300 and 900 m²/g.

Both removal of the organic template and the decomposition of ammonium-ions present form acid sites in the zeolite catalyst. These acid sites are active in dimerization and oligomerization.

According to another preferred embodiment of the invention, acid sites in the catalysts are formed by ion exchange with protons in a liquid Bronsted acidic medium. The catalyst exhibits Bronsted acid sites.

According to an embodiment of the invention, acid sites are formed through hydrolysis of hydration water by polyvalent cations.

The chemical composition of the zeolite and the mesoporous aluminosilicate materials can vary depending on the original composition of preparation method and treatment performed after preparations. Common treatments include vapour treatments and acid and silicon tetrachloride treatments for dealumination, ion exchange treatments for modification of pore size and acidity, impregnations and gas phase treatments for introducing metals on the surface.

Zeolite or mesoporous aluminosilicate catalysts in use comprise a zeolite or mesoporous aluminosilicate and a carrier. Suitable carriers are for example silica, alumina, clay or any mixture of these. The carrier serves to give formability, hardness and in some cases additional, suitable activity to the dimerization reaction. The catalyst can contain about 10 to 100 wt-% active material, the rest of the catalyst being the carrier.

According to the first preferred embodiment of the present dimerization process, C₄ olefins are dimerized. The compositions of the feed have already been discussed, and product compositions then are as follows:

The dimer fraction of the reaction product for a feed comprising (among other, less reactive compounds) both C₄ and C₅ isoolefins (at a ratio of 45 to 55) includes trimethylpentenes in a concentration of 20 to 30 wt-%, in particular 25 to 28 wt-%, tetramethylpentenes and trimethylhexenes in a concentration of 20 to 30 wt-%, in particular 20 to 25 wt-%, tetramethylhexenes in a concentration of 4 to 8 wt-%, in particular 5 to 6 wt-%, and trimethylheptenes in a concentration of 2 to 5 wt-%, in particular 3 to 4 wt-%. The rest of the oligomer product is less branched olefins.

When mainly dimers of isobutene are produced, they are typically present in the product flow in at least 85 wt-%, preferably at least 90 wt-%. Other components typically present in the product flow are trimers of isobutene, 15 wt-% or less, preferably 10 wt-% or less, tetramers of isobutene in less than 0.2 wt-% and other hydrocarbons in less than 1 wt-% preferably less than 0.1 wt-%.

Regardless of the aimed product composition most (65 to 100 wt-%, typically 85 to 100 wt-%, preferably 95 to 100 wt-%) of the dimers produced by the process are 2,4,4-trimethyl pentenes. When the product stream is hydrogenated, a mixture comprising isooctane is obtained. The fraction of other trimethyl pentanes (e.g. 2,3,4-trimethyl pentane) as well as the fraction of dimethyl hexanes in the mixture remains extremely small. Thus the octane number (RON) of the fuel component is high, typically at least 95, preferably approximately 98 to 100.

According to the second preferred embodiment, dimers of C₅ olefins are produced. The product is typically as follows:

At least 65 wt-%, preferably at least 70 wt-%, C₅ dimers, 5 to 32 wt-%, preferably 5 to 29 wt-% olefin trimers, less than 1 wt-%, preferably less than 0.5 wt-% olefin tetramers. Because no oxygenate is fed to the process, the amount of oxygenates in the process and in the final product is very small. When the composition is hydrogenated, a composition useful as a fuel component is obtained.

According to the third embodiment, dimers of both C₄ and C₅ olefins are produced. In addition also C₄ and C₅ olefins react and form C₉ olefins. The product composition then comprises at least 65 wt-%, preferably at least 70 wt-%, C₅ dimers, C₄ dimers and C₉ olefins, 5 to 32 wt-%, preferably 5 to 28.5 wt-% olefin trimers, less than 1 wt-%, preferably less than 0.5 wt-% olefin tetramers. When the composition is hydrogenated, a composition useful as a fuel component is obtained.

Regardless of the aimed product composition most (50 to 100 wt-%, typically 60 to 100 wt-%, preferably 90 to 100 wt-%) of the dimers and C₉ olefins produced by the process are isooctene, tetramethylpentenes and trimethylhexenes. When the product stream is hydrogenated, a mixture comprising corresponding hydrogenated hydrocarbons is obtained. The relative abundance of individual components varies depending on the ratio of the reactive C₄ and C₅ components in the feed. When the product stream is hydrogenated, a mixture comprising isooctane, tetramethylpentanes and trimethylhexanes is obtained. Thus the octane number (RON) of the fuel component is high, typically at least 95, preferably approximately 98 to 100.

The dimer fraction of the reaction product for a feed comprising (among other, less reactive compounds) both C₄ and C₅ isoolefins (at a ratio of 45 to 55) includes trimethylpentenes 20 to 30 wt-%, in particular 25 to 28 wt-%, tetramethylpentenes and trimethylhexenes 20 to 30 wt-%, in particular 20 to 25 wt-%, tetramethylhexenes 4 to 8 wt-%, in particular 5 6 wt-%, and trimethylheptenes 2 to 5 wt-%, in particular about 3 to 4 wt-%. The rest of the dimer product is formed by less branched olefins.

The product has a vapour pressure of 10 to 20 kPa and a distillation point (90 vol-%, ASTM D86) is equal or less than 180° C.

According to an embodiment, a part of the oligomerized product, which is not recycled, is transferred to alkylation.

According to an embodiment, the oligomerized product is subjected to hydrogenation to provide a partly or totally hydrogenated product.

Preferred process configurations are presented in the following.

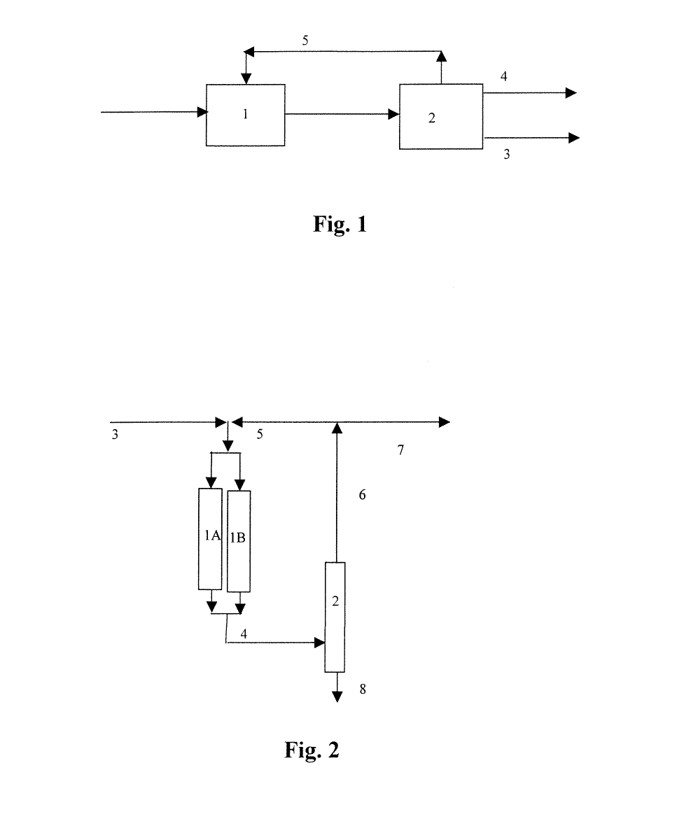

According to one embodiment of the invention, the process comprises a reaction zone 1 and a separation zone 2, as presented in FIG. 1. The product is formed in the reaction zone and in the separation zone the product is separated from unreacted components in flow 3. The inert components and the remaining feed leave the process in flow 4. The remaining feed and solvent is returned to the process along with flow 5. The figure is schematic and for simplicity only one recycle stream is shown. However it should be understood that in actual plant solvent recycle may be accomplished via a line separate from the remaining feed or via the same line, depending on the structure of the separation zone. This remark applies to all drawings.

The reaction zone comprises one or several reactors. Many reactors of a continuous type capable of housing a solid catalyst and a liquid reagent are suitable for the invention. According to an embodiment of the invention, the reactor must allow regeneration of the catalyst. The regeneration can be done during continuous process operation. Alternatively, two or several reactors can be used in parallel, this allows regenerating one reactor when other is being operated.

A typical oligomerization system comprises one or more reaction sections followed by product separation and arrangements for recycling of the unreacted reactants and solvent. Several reaction and product separation stages may be connected in series if conversion requirement is high.

According to an embodiment of the invention, the reaction zone comprises any reactor type suitable for liquid phase operation and in which a solid catalyst can be used. These reactor types include a fixed bed reactor, a moving bed reactor, a mixing tank reactor, a fluidized bed reactor, or a spouted bed reactor or a combination of these reactors.

In conventional processes, in order to meet the requirements for continuous operation, the dimerization catalyst must be regenerated regularly, even on a daily basis. By contrast, in the present invention, catalyst life is prolonged and the need for regeneration is strongly reduced.

However, even in the invention, there can be a need for occasional regeneration of the catalyst. For this purpose, the process can be complemented with facilities for catalyst regeneration in the reactor system. If continuous operation is not imperative, it is of course possible to pause process operation for catalyst regeneration. However, in industrial operation it is preferred to have several reactors that can be regenerated one at a time, while the others are in production. An example of such an arrangement is two or more fixed bed reactors connected in such a manner that each of them can be separated from the process for changing or regenerating the catalyst.

Another option is to use a reactor from which the catalyst can be extracted continuously for regeneration. For this purpose, a fluidized bed or spouted bed reactor can used, from which the catalyst can be extracted continuously and recycled through a regeneration facility.

According to a preferred embodiment of the invention, the separation zone comprises a distillation column. The product flow from the reaction zone comprises light hydrocarbons remaining from the hydrocarbon feed, and oligomers formed in the reactor having a boiling point substantially higher than that of the feed. This makes separation by distillation simple.

According to an embodiment of our invention, the separation zone is preferably a distillation zone. The reactants are monomers and the product is a mixture of oligomers and thus they have significantly different boiling points making separation by distillation easy. Considering the ease of separation, a flash drum, evaporator, stripper, or fractionator and other distillation devices known in the art can be used. Thus, as pointed out above, the separation zone may comprise also absorption, adsorption, membrane separation or extraction stages.

Another preferred embodiment of the invention is presented in FIG. 2. The reaction zone comprises two reactors in parallel 1A and 1B used in turn. This means that when one reactor is being regenerated, the other reactor is used for the dimerization. The separation zone comprises a distillation column 2. Flow 8 comprises the product flow leaving from the separation zone. The flow 6 at the top of the distillation column comprises the unreacted feed. A part of the feed 7 is withdrawn from the process and the other part is directed back to the separation zone in order to raise the yield of the reaction zone.

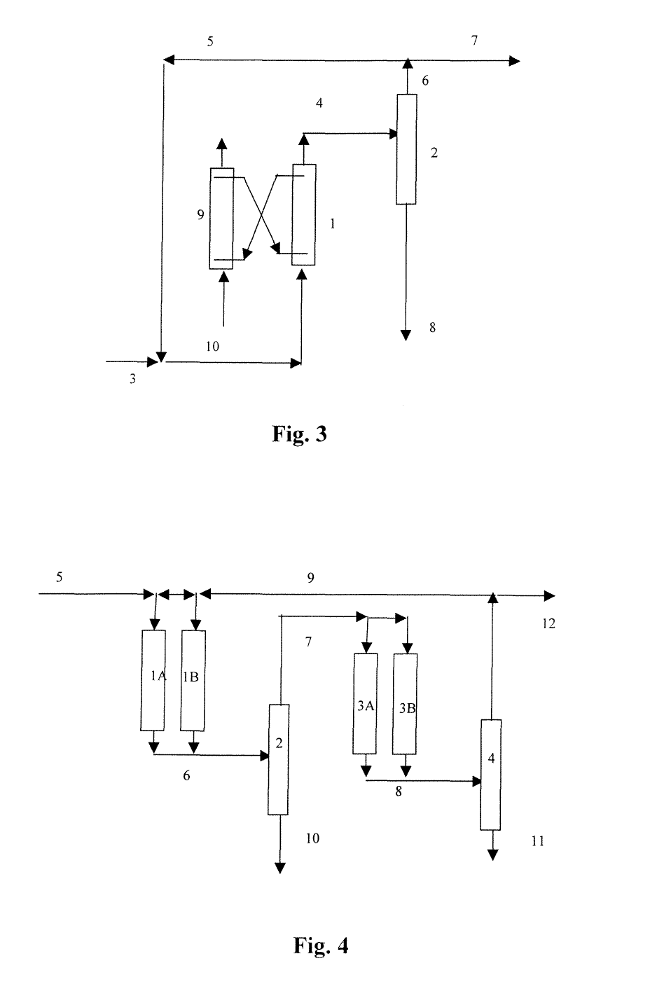

Another advantageous embodiment of our invention is presented in FIG. 3. In this embodiment, the reactor is a fluidized bed reactor and the catalyst is continuously regenerated in a regenerator unit 9.

FIG. 4 presents another preferred embodiment of the invention. The yield of the reaction zone is improved by connecting two reaction zones. Both reaction zones comprise two reactors in parallel, i.e. 1A/1B and 3A/3B. In the first separation zone 2, the dimer formed in the first reaction zone is separated in flow 10 from the unreacted components in flow 7, and in the second separation zone 4 the dimer formed in the second reaction zone is separated in flow 11 from the unreacted components in flow 12. Some of the unreacted components are returned to the feed in flow 5 via flow 9.

### EXAMPLES

### Examples 1-7

Target of these examples is to show the superprior properties of supercritical carbon dioxide as a solvent in the isobutene dimerization.

Isobutene was dimerized continuously in a stirred tank reactor at temperature 100° C. Two different feed compositions were used. Isobutene content of feed was approximately 30%. n-Hexane (1 wt-%) was used as an internal standard. Carbon dioxide or propane acted as a reaction medium. Propane were chosen as a comparative solvent for carbon dioxide since it has the similar molecular weight. The content of solvent was approximately 70%. The pressure of the tests were chosen based on the calculated phase envelopes. The pressure in tests made with the supercritical carbon dioxide (ex 1, 3, 5) was 8.9 MPa. The pressure in tests made with the supercritical propane was 4.9 MPa (ex 2, 4, 6). The pressure of test made in carbon dioxide but in mixed phase (ex 7) was 45 bar.

The catalyst used in examples 1, 2 and 8 was commercial ZSM-5. The catalyst used in examples 3, 4 and 7 were prepared based WO patent application 2004/080590. MCM-41 was obtained from Abo Akademi University. All the catalysts were in acid form.

The results of isobutene tests made with supercritical carbon dioxide (inventive examples) and with supercritical propane (comparative examples), and with mixed phase (comparative example) are summarized in Table 1. The deactivation rates of catalysts are essential smaller in supercritical carbon dioxide than in supercritical propane or in mixed phase.

TABLE 1
Summary of isobutene reaction tests in supercritical carbon
dioxide and in supercritical propane.

| | | | | | Con- | Se- | |
|---|---|---|---|---|---|---|---|
| | | | | | version | lec- | Selec- |
| | | | | Run | of | tivity | tivity |
| | | | | time | isobutene | to C8 | to C12+ |
| | Catalyst | Solvent | WHSV | (h) | (%) | (%) | (%) |
| Ex 1 | ZSM-5 | scCO2 | 22 | 10 | 60 | 64 | 35 |
| | | | | 100 | 26 | 89 | 11 |
| Ex 2 | ZSM-5 | scPropane | 25 | 10 | 28 | 91 | 8 |
| | | | | 100 | 9 | 97 | 2 |
| Ex 3 | ZSM-23 | scCO2 | 16 | 10 | 83 | 34 | 51 |
| | | | | 100 | 65 | 57 | 43 |
| Ex 4 | ZSM-23 | scPropane | 12 | 10 | 70 | 60 | 40 |
| | | | | 100 | 36 | 85 | 13 |
| Ex 5 | MCM- | scCO2 | 60 | 10 | 94 | 9 | 91 |
| | 41 | | | 200 | 81 | 25 | 75 |
| Ex 6 | MCM- | scPropane | 50 | 10 | 87 | 22 | 77 |
| | 41 | | | 200 | 63 | 61 | 39 |
| Ex 7 | ZSM-23 | CO2 | 17 | 6 | 38 | 63 | 37 |
| | | (mixed | | | | | |
| | | phase) | | | | | |

Test results shows the superior properties of supercritical carbon dioxide as a reaction medium in isobutene dimerization for the diminishing of the deactivation of the acidic aluminosilicate catalysts.

### Examples 8-13

Target of these examples is to show the superprior properties of supercritical carbon dioxide as a medium for regeneration of catalyst.

ZSM-5 catalyst was deactivated in isobutene dimerization test at 100° C. in 89 bar with WHSV 35 h⁻¹. After deactivation, the catalyst was treated with supercritical carbon dioxide for removal of coke. Then, the reaction of isobutene was started again to see if the regeneration of catalyst was successful. The comparative regeneration was performed with the supercritical propane. At 200° C., propane started to react, and the regeneration with the presence of propane was impossible. Table 2 summarises the results.

TABLE 2
Summary of regeneration tests in supercritical carbon dioxide and
in supercritical propane. The conversion values are the conversion
of isobutene after the regeneration.

| | | | Conversion |
|---|---|---|---|
| Examples | | Regeneration method | (%) |
| | Fresh | | 64 |
| | catalyst | | |
| | Deactivated | | 13 |
| | catalyst | | |
| Ex 8 | Regenerated | scCO2 100° C., 25 h-1, 24 h | 16 |
| Ex 9 | Regenerated | scCO2 200° C., 25 h-1, 48 h | 50 |
| Ex 10 | Regenerated | scCO2 200° C., 25 h-1, 24 h | 41 |
| Ex 11 | Regenerated | scCO2 200° C., 25 h-1, 24 h | 42 |
| Ex 12 | Regenerated | scCO2 250° C., 25 h-1, 24 h | 67 |
| Ex 13 | Regenerated | scPropane, 100° C., 25 h-1, 24 h | 8 |

As shown in examples, the supercritical carbon dioxide regenerated the catalysts at low temperatures compared with the temperatures used in the conventional regenerations.

1. A process for oligomerizing olefinic, lower hydrocarbons, comprising:
feeding a fresh olefinic hydrocarbon feedstock to a reaction zone;
contacting the olefinic hydrocarbons of the feedstock in a homogeneous phase with an acidic catalyst in the reaction zone in order to dimerize at least a part of the olefinic hydrocarbons, wherein the acidic catalyst is selected from the group of natural and synthetic zeolites or from the mesoporous aluminosilicates;
withdrawing an effluent containing oligomerized olefins from the reaction zone; and
conducting the effluent to a separation zone, wherein the oligomerization reaction product is separated from said effluent;
wherein the homogeneous phase comprises a solvent for olefinic hydrocarbons maintained at supercritical conditions or at liquid phase.

2. The process according to claim 1, wherein the solvent is selected from the group consisting of nitrogen, methane, trifluoromethane, carbon dioxide, ethane, nitrooxidule, sulphur hexafluoride, difluoromethane, ammonia, isobutane and water and mixtures thereof.

3. The process according to claim 2, wherein the solvent is nitrogen or carbon dioxide.

4. The process according to claim 1, wherein the olefinic hydrocarbons are fed to the reaction zone in a homogeneous phase that can be liquid or supercritical.

5. The process according to claim 1, wherein the olefinic hydrocarbons and the solvent are maintained in supercritical phase during dimerization.

6. The process according to claim 1, wherein the homogeneous phase comprises carbon dioxide maintained at supercritical conditions.

7. The process according to claim 1, wherein the solvent is carbon dioxide having a purity of about 50 to 100% by weight.

8. The process according to claim 1, wherein the olefinic hydrocarbons and the solvent are maintained at a pressure of 15 to 200 bar.

9. The process according to claim 8, wherein the temperature of the supercritical phase is 300-420 K.

10. The process according to claim 1, wherein the molar ratio of solvent to the olefinic hydrocarbon feed is about 100:1 to 1:100, preferably about 10:1 to 1:10, in particular about 8:1 to 1:1.

11. The process according to claim 1, wherein the oligomers of the oligomerization reaction product mainly comprises dimers selected from the group of C₆ to C₁₂ olefinic hydrocarbons.

12. The process according to claim 1, wherein the oligomers of the oligomerization reaction product comprises trimers selected from the group of C₉ to C₁₈ olefinic hydrocarbons.

13. The process according to claim 1, wherein the zeolite is selected from the group consisting of synthetic and natural zeolites containing about 0.1 to 5 wt-% of aluminum.

14. The process according to claim 13, wherein the zeolite is selected from the group consisting of ZSM-5, ZSM-22, ZSM-23, ferrierite and ion-exchanged zeolites prepared therefrom.

15. The process according to claim 13, wherein the zeolite is selected from the group consisting of beta, Y-zeolite and ion-exchanged zeolites prepared therefrom.

16. The process according to claim 13, wherein the catalyst is a mesoporous aluminosilicate which is selected from the group consisting of synthetic and natural aluminosilicates having a regular or irregular pore system.

17. The process according to claim 16, wherein the catalyst is a mesoporous aluminosilicate having a regular pore system, such as MCM-41, or an amorphous mesoporous aluminosilicate having an irregular pore system.

18. The process according to any of claims 13 to 17, wherein the catalyst contains aluminium about 0.1 to 50 wt-%, preferably about 2 to 10 wt-%, and has a BET surface areas of 200 to 1000 m²/g, preferably 300 to 900 m²/g.

19. The process according to claim 1, wherein the catalyst exhibits Bronsted acid sites.

20. The process according to claim 13, wherein the olefins present in the olefinic feedstock are selected from the group of isobutene, 1-butene, 2-butene, linear and branched C₅-olefins.

21. The process according to claim 20, wherein the feedstock comprises 10 to 100 wt-% of isobutene.

22. The process according to claim 1, wherein the olefins present in the olefinic feedstock are selected from the group of linear and branched C₅-olefins.

23. The process according to claim 1, comprising combining a recycled product withdrawn form the separation zone with the fresh feed to form a combined feed of olefins for the reaction zone.

24. The process according to claim 23, wherein the flow of the recycled product is 20 to 150 wt-%.

25. The process according to claim 1, wherein the solvent is separated from the effluent by lowering of the pressure.

26. The process according to claim 1, wherein the dimerization reaction is carried essentially in the absence of polar compounds.

27. The process according to claim 26, wherein the amount of polar compounds is less than 0.5 mole-%, preferably less than about 0.2 mole-%, of the olefinic hydrocarbons fed into the reaction zone.

28. The process according to claim 1, wherein the oligomerization products is partly or totally hydrogenated.

29. The process according to claim 1, wherein the separation zone comprises a flash distillation zone.

30. The process according to claim 1, wherein the oligomerization process is carried out in a reactive distillation system including at least one reaction zone and at least one distillation zone, said at least one reaction zone including at least one reactor and said at least one distillation zone including at least one distillation column.

31. The process according to claim 1, wherein isooctene is produced from a feed comprising isobutene, and the isooctene is optionally hydrogenated to yield isooctane.

32. The process according to claim 1, wherein the catalyst is regenerated by heating it in a regeneration medium comprising 50 to 100% carbon dioxide, said percentage being calculated from the weight of the medium.

33. The process according to claim 32, wherein the regeneration medium comprises spent reaction solvent.

34. The process according to claim 32 or 33, wherein regeneration is carried out at temperature below 500° C., preferably at about 100 to 300° C.

35. Use of carbon dioxide in supercritical phase as a solvent for dimerization of lower isoolefins.
